(19) United States
(12) Patent Application Publication
(10) Pub. No.: US 2019/0380820 A1
(43) Pub. Date: Dec. 19, 2019

(54) METHOD AND DEVICES FOR FASCIA CLOSURE

(71) Applicant: Gilbertas Rimkus, Myrtle Beach, SC (US)

(72) Inventor: Gilbertas Rimkus, Myrtle Beach, SC (US)

(21) Appl. No.: 16/008,527

(22) Filed: Jun. 14, 2018

(51) Int. Cl. A61F 2/00; A61B 17/00; A61B 17/04

(52) U.S. Cl. CPC A61F 2/0063; A61B 17/00234; A61B 2017/00004; A61F 2002/0072; A61B 17/0483

(57) ABSTRACT

A surgical mesh having a pocket for housing and retaining at least one ribbon retractor.

### RELATED APPLICATIONS

This application is related to U.S. Provisional Patent Application No. 62/528721, filed on Jul. 5, 2017.

### FIELD OF THE INVENTION

The present application discloses and describes a method and devices for closing an incision.

### BACKGROUND OF THE INVENTION

Surgical mesh patches used in the closure of an abdominal incision are often closed using either a mass closure technique (one suture for all layers) or a layered closure technique (multiple sutures across the multiple layers). In layered closure, multiple separate sutures are used. Most often, each layer of incision is closed using a separate suture. An advantage of using layered closure with separate, multiple suture strands is that if one of the suture strands breaks or loosens, the remaining suture strands maintain the remaining incision closures and integrity of the sutured-field.

The abdominal fascia is a membrane of connective tissue that lies between the inner surface of the transverse abdominal muscle and the parietal peritoneum and lines the abdominal parietes. Surgical procedures to correct or manage hernias and other similar organ prolapse have been developed and used for decades. One means to better correct and/or manage organ prolapse includes the development and usage of surgical mesh to support such organs. Over time, a variety of absorbable and non-absorbable surgical meshes have been developed and continue in use.

A possible consequence of such fascial surgical repairs includes incisional hernia, which may develop as a result of an incomplete repair or healing of a surgical incision. And because post-surgical incisional hernias can occur even months and years after the original surgical repair, this is an on-going, long-term concern for both surgeon and patient.

Thus, as cases may warrant, additional post-surgery procedure(s) may be necessary to repair and/or manage an incisional hernia springing from an original or subsequent surgery. Unfortunately, incisional hernia issues are a costly problem with regard to costs, resources, inconvenience, patient satisfaction, and ideal post-operative recuperation and/or management. For example, recent data for 2006 demonstrates that approximately 340,000 incisional hernia repairs were performed in the United States at a cost of approximately $3.2 billion to patients and the health-care system.

Accordingly, there is a need for an improved surgical technique utilizing improved structure and elements of surgical mesh.

A search of the prior art did not disclose any patents that read directly on the claims of the instant invention; however, the following references were considered related:

U.S. Pat. No. 8,357,172 B2, issued in the name of Harper;

U.S. Pat. No. 6,171,318 B1, issued in the name of Kugel et al.;

U.S. Pat. No. 6,280,453 B1, issued in the name of Kugel et al.;

U.S. Patent Application no. 2010/0185219 A1, published in the name of Gertzman et al.;

U.S. Patent Application no. 2013/0006280 A1, published in the name of Adams;

U.S. Pat. No. 8,945,235 B2, issued in the name of Horton et al.;

U.S. Pat. No. 8,562,633 B2, issued in the name of Cully et al.;

U.S. Patent Application no. 2004/0019360 A1, published in the name of Farnsworth et al.; and

U.S. Patent Application no. 2010/0241145 A1, published in the name of Cook.

This application presents claims and embodiments that fulfill a need or needs not yet satisfied by the devices, inventions and methods previously or presently available. In particular, the claims and embodiments disclosed herein describe a surgical mesh, the surgical mesh comprising a pair of long edges and a pair of short edges, wherein one of the long edges comprises a plurality of apertures, and wherein at least one pocket is formed within the mesh between the long edges and short edges, the at least one pocket accommodating at least one removable stainless-steel ribbon retractor, the surgical mesh providing unanticipated and nonobvious combination of features distinguished from the devices, inventions and methods preexisting in the art. The applicant is unaware of any device, method, disclosure or reference that discloses the features of the claims and embodiments disclosed herein.

### SUMMARY

It is envisioned that the embodiments disclosed herein provide a surgical mesh and at least one ribbon retractor apparatus utilized in a method of closing a surgical incision, wherein the mesh comprises a polyglycolic acid mesh that is designed to prevent incisional hernia. Such methods and/or techniques and apparatuses provide an improved means for reducing the frequency and severity of incisional hernias, thereby reducing health care costs and improving short and long-term patient outcomes. Additionally, the disclosed methods/techniques and apparatuses, separately and/or in combination, further provide protection to exposed organs, vessels, and tissues, in that the mesh and ribbon retractors provide a barrier between the surgical instruments and the organs, vessels, and/or tissues exposed during surgery. Moreover, the disclosed methods/techniques and apparatuses, separately and in combination, provide for a method or technique that is simplified so that surgical competency and excellent are quickly achieved, as well as a technique that reduces the amount of time necessary to repair and close an incisional hernia, further saving costs and reducing patient exposure to environmental pathogens or dangers.

In accordance with one embodiment, a surgical mesh comprising a pair of long edges and a pair of short edges, wherein one of the long edges comprises a plurality of apertures, and wherein at least one pocket is formed within the mesh between the long edges and short edges, the at least one pocket accommodating at least one removable stainless-steel ribbon retractor.

In accordance with another embodiment, a surgical mesh comprising a top layer and a base layer, wherein the top layer includes, a first slit providing ingress and egress to a first sleeve, and a second slit providing ingress and egress to a second sleeve, and each one of the first sleeve and the second sleeve separately housing an insertable and removable stainless-steel ribbon retractor.

In either embodiment, the ribbon retractor comprises a linearly elongated body having mutually opposed long-sides and mutually opposed short-sides forming a generally flat and linear body. It is further envisioned that one of the short-sides of the ribbon retractor includes a slight upward bend. It is further envisioned that one of the short-sides of the ribbon retractor includes an aperture. It is further envisioned that one of the short-sides of the ribbon retractor includes a slight upward bend and an aperture.

### BRIEF DESCRIPTION OF THE DRAWING(S)

FIG. 1 is a block diagram of the general steps of a surgical method described herein utilizing a surgical mesh with at least one pocket that houses and/or retains at least one ribbon retractor;

FIG. 2 is a sectional-view of the abdomen illustrating the multiple layers and the use of an improved surgical mesh as part of the method described herein to suture and close an abdominal incision;

FIG. 3 is an expanded plan view;

FIG. 4a through 4e are multiple views of an improved surgical mesh having at least two layers and forming at least one sleeve or multiple sleeves to receive one or more stainless-steel ribbon retractors;

FIG. 5 is a view depicting a version of the surgical mesh wherein multiple stainless-steel ribbon retractors are inserted through the sleeves of the surgical mesh before placement and implantation within the abdomen during surgery; and

FIG. 6 is a plan view of a plurality of ribbon retractor embodiments of different dimensions that may be utilized.

### DESCRIPTION OF THE EMBODIMENT(S)

It will be readily understood that the components of the present invention, as generally described and illustrated in the figures herein, may be arranged and designed in a wide variety of different configurations. Thus, the following detailed description of the embodiments, as represented in the attached figures, is not intended to limit the scope of the invention as claimed but is merely representative of selected embodiments of the invention.

The features, structures, or characteristics of the invention described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

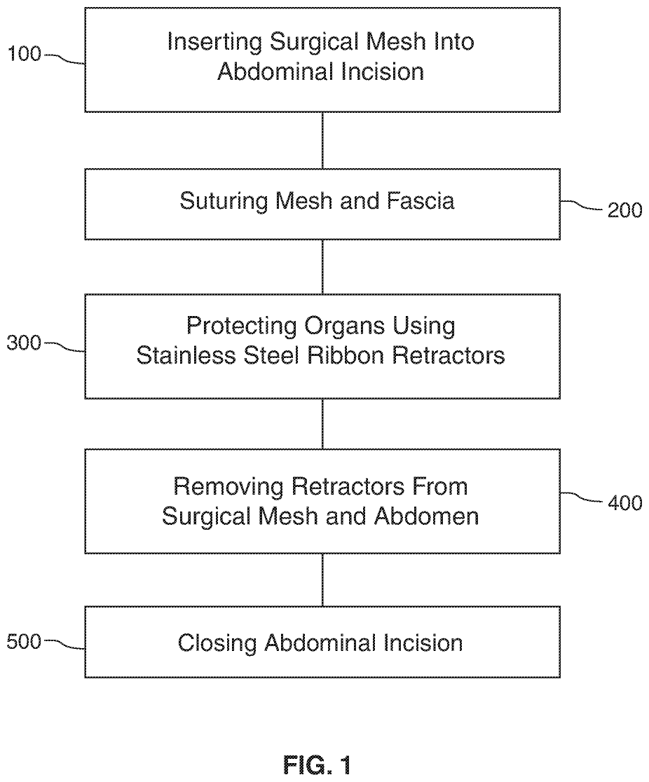

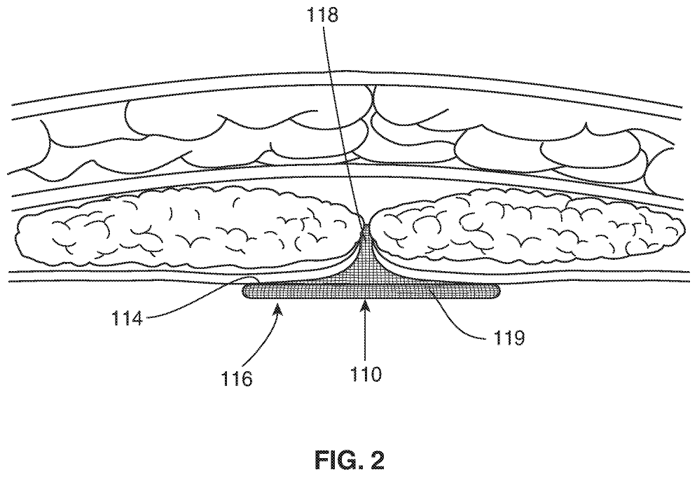

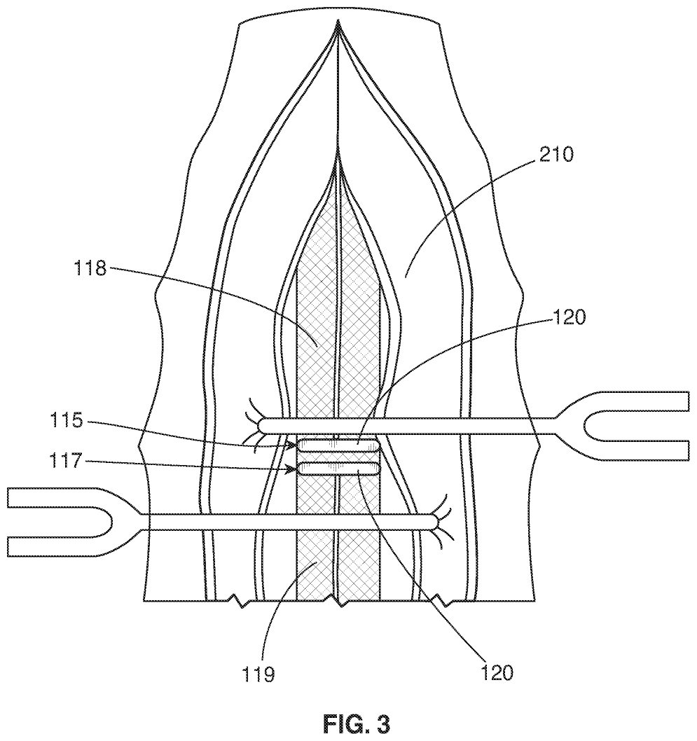

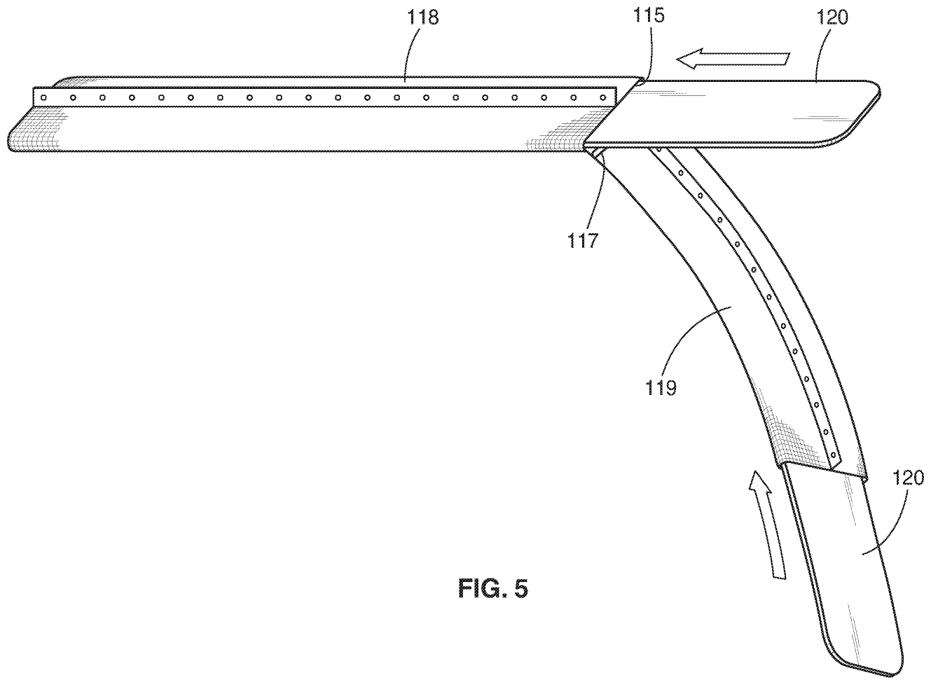

In accordance with the drawings illustrating at least one embodiment of an improved surgical mesh and a technique incorporating such improved surgical mesh, as generally depicted in FIG. 1 through FIG. 6. Consistent with FIG. 1, a method of surgically closing an abdominal incision may be generally described as comprising the steps of inserting 100 surgical mesh 110, the surgical mesh 110 having a plurality of removable stainless-steel retractors 120 into the abdominal incision. The method further includes the step of suturing 200 the surgical mesh 110 and the fascia 210 together using two suturing strands. The method further includes the step of protecting 300 the organs, tissues, and blood vessels subjacent the implanted surgical mesh 110 from contact by the suturing needle via the stainless-steel ribbon retractors 120 that deflect such contact by or from the suturing needle. Thereafter, the method calls for removing 400 the stainless-steel ribbon retractors 120 from the surgical mesh 110. The method may conclude with the closing 500 of the abdominal incision by joining the remaining portions of the individual sutures.

It is envisioned that the surgical mesh 110 utilized is an absorbable mesh made from polyglycolic acid or similar material. In one embodiment, the polyglycolic surgical mesh 110 may include a plurality of apertures 112 formed along one edge 111 of the mesh 110. In particular, it is envisioned that the plurality of apertures 112 may be formed or disposed along the longest-edge 111 of the mesh.

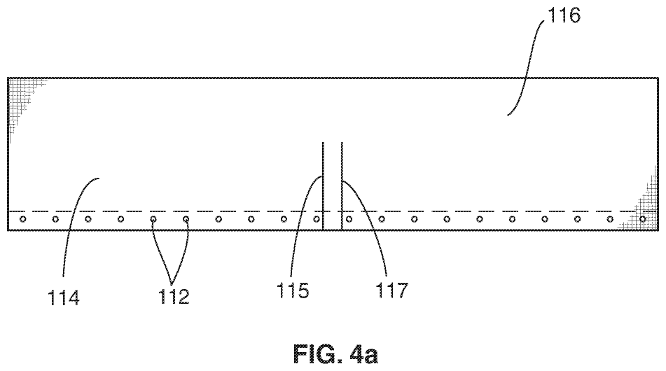

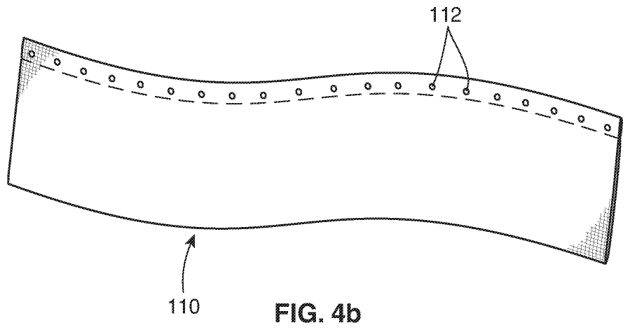

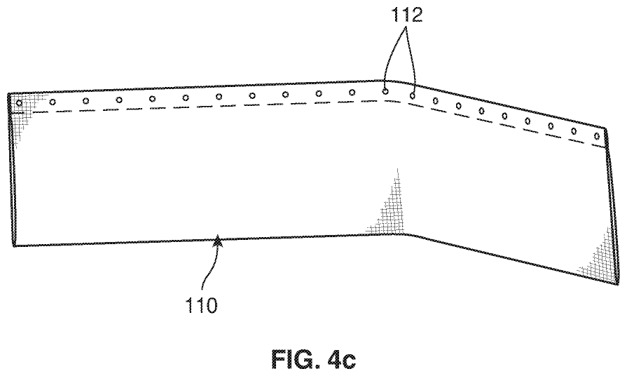

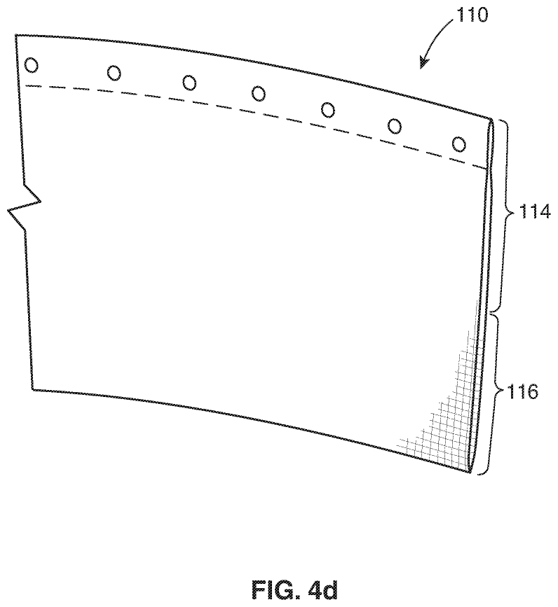

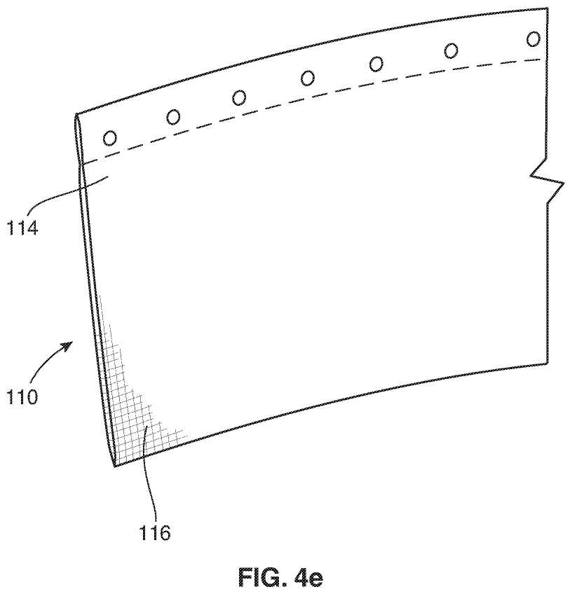

Consistent with FIGS. 4a through 4e, it is also envisioned that the mesh 110 includes at least two layers, thereby comprising a top layer 114 and a base layer 116. The top layer 114 includes a first slit 115 providing ingress and egress to a first sleeve 118 and a second slit 117 providing ingress and egress to a second sleeve 119, each one of the first sleeve 118 and the second sleeve 119 separately housing an insertable and removable stainless-steel ribbon retractor 120.

During the suturing process (generally denoted by 200 in FIG. 1), the stainless-steel ribbon retractor 120 acts to protect (generally denoted by 300 in FIG. 1) the organs, tissues, and/or blood vessels subjacent to the implanted surgical mesh 110. The ribbon retractor 120 may be a linearly elongated body having mutually opposed long-sides and mutually opposed short-sides forming a generally flat and linear body in the form similar to that of a fingernail file. It is also envisioned that one end of the ribbon retractor 120 (preferably one of the short-ends) may include a slight upward bend to assist in grasping and removal during step 400. It is also envisioned that the same end of the ribbon retractor 120 may include an aperture to assist in grasping and removal during step 400. It is also envisioned that the combination of the slight upward bend and the aperture may be included in the same end of the ribbon retractor 120 to further facilitate removal of the ribbon retractor(s) 120 from the sleeve(s) 118 and/or 119 during removal step 400.

The ribbon retractor 120 comprises stainless-steel to provide for ease of sterilization. Moreover, the ribbon retractor 120 comprises stainless-steel to provide for sufficient rigidity and durability during the insertion, suturing, and removal processes. Once inserted into the mesh 110 (via sleeve 118 or sleeves 118 and 119), the stainless-steel ribbon retractor 120 acts as a shield between the mesh 110 and the organs, tissues, and/or blood vessels subjacent the implanted mesh 110 and the barbed-end of the suturing needle. Because the barbed-end of the suturing needle cannot penetrate or otherwise pierce the ribbon retractor 120, the abdominal incision may be sutured (200) with better precision and speed, reducing incisional herniation as well as opportunity for environmental contamination or other such exposures that are inherent with such incisional-based procedures.

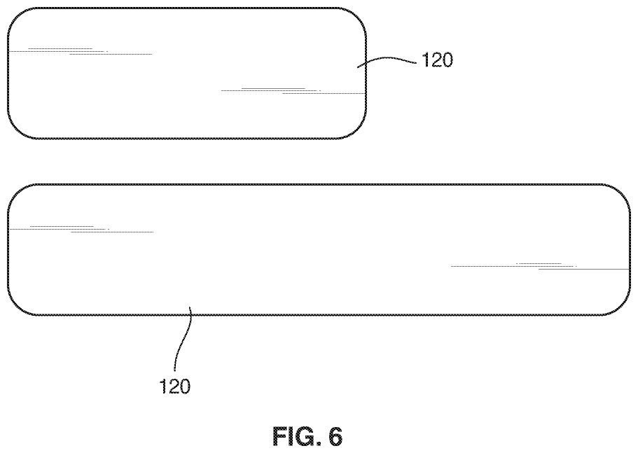

As particularly depicted in FIG. 6, at least two embodiments of the ribbon retractor 120 are depicted, the two embodiments illustrating different sizes that are envisioned. As but one example, one embodiment of the ribbon retractor 120 comprises a short-edge having a length of approximately 4 cm and a long-edge having a length of approximately 10 cm. Because the retractor 120 is a four-edged component, the opposing short-edge and opposing long-edge are substantially similar in length, thereby forming a rectangular shape viewing the retractor 120 from a plan view. In another example as shown, the short-edge comprises a length of approximately 4 cm and the long-edge comprises a length of approximately 15 cm, with the respective opposing edges having substantially similar lengths and consistent with the description of the first retractor 120 embodiment above. It is further envisioned that other dimensions are possible, including slightly shorter lengths for the short-edge so long as the shorter length provides the requisite protective shield of the abdominal field, and slightly longer lengths for the short-edge so long as the longer length does not exceed the width of the surgical mesh, as well as slightly shorter and/or longer lengths of the long-edge.

Near completion of closing the abdominal incision, any remaining stainless-steel ribbon retractor(s) 120 is/are removed from the implanted surgical mesh 110. Removing the stainless-steel ribbon retractors 120 from the surgical mesh 110 (generally denoted by 400 in FIG. 1) requires grasping one or more of the retractors by hand or using an instrument and then sliding the ribbon retractor 120 from the sleeve 118 or 119 in which the ribbon retractor 120 is housed.

To complete suturing and closing of the abdominal incision (generally denoted by step 500 in FIG. 1), the sutures are tied together in a closing knot and the excess length of each strand are clipped and removed from the surgical field. Thereafter, the remaining exterior layers of muscle/fat and/or skin may be closed using additional sutures, including different gauges of suture and/or staples separately or in combination.

It is to be understood that the embodiments and claims are not limited in application to the details of construction and arrangement of the components set forth in the description and/or illustrated in drawings. Rather, the description and/or the drawings provide examples of the embodiments envisioned, but the claims are not limited to any particular embodiment or a preferred embodiment disclosed and/or identified in the specification. Any drawing figures that may be provided are for illustrative purposes only, and merely provide practical examples of the invention disclosed herein. Therefore, any drawing figures provided should not be viewed as restricting the scope of the claims to what is depicted.

The embodiments and claims disclosed herein are further capable of other embodiments and of being practiced and carried out in various ways, including various combinations and sub-combinations of the features described above but that may not have been explicitly disclosed in specific combinations and sub-combinations.

Accordingly, those skilled in the art will appreciate that the conception upon which the embodiments and claims are based may be readily utilized as a basis for the design of other structures, methods, and systems. In addition, it is to be understood that the phraseology and terminology employed herein are for the purposes of description and should not be regarded as limiting the claims.

What is claimed is:

1. A surgical mesh comprising a pair of long edges and a pair of short edges, wherein one of the long edges comprises a plurality of apertures, and wherein at least one pocket is formed within the mesh between the long edges and short edges, the at least one pocket accommodating at least one removable stainless-steel ribbon retractor.

2. The surgical mesh of claim 1, wherein the ribbon retractor comprises a linearly elongated body having mutually opposed long-sides and mutually opposed short-sides forming a generally flat and linear body.

3. The surgical mesh of claim 2, wherein one of the short-sides of the ribbon retractor includes a slight upward bend.

4. The surgical mesh of claim 3, wherein one of the short-sides of the ribbon retractor includes an aperture.

5. The surgical mesh of claim 2, wherein one of the short-sides of the ribbon retractor includes a slight upward bend and an aperture.

6. A surgical mesh comprising:
a top layer and a base layer, wherein the top layer includes,
a first slit providing ingress and egress to a first sleeve, and
a second slit providing ingress and egress to a second sleeve, and wherein
each one of the first sleeve and the second sleeve separately housing an insertable and removable stainless-steel ribbon retractor.

7. The surgical mesh of claim 6, wherein the ribbon retractor comprises a linearly elongated body having mutually opposed long-sides and mutually opposed short-sides forming a generally flat and linear body.

8. The surgical mesh of claim 7, wherein one of the short-sides of the ribbon retractor includes a slight upward bend.

9. The surgical mesh of claim 7, wherein one of the short-sides of the ribbon retractor includes an aperture.

10. The surgical mesh of claim 7, wherein one of the short-sides of the ribbon retractor includes a slight upward bend and an aperture.

11. The surgical mesh of claim 6, wherein one of the retractors comprises a short-edge of approximately 4 cm and a long-edge of approximately 10 cm.

12. The surgical mesh of claim 6, wherein one of the retractors comprises a short-edge of approximately 4 cm and a long-edge of approximately 15 cm.
